(19) United States
(12) Patent Application Publication
(10) Pub. No.: US 2007/0197817 A1
(43) Pub. Date: Aug. 23, 2007

(54) Method for the preparation of 3-substituted-3'-hydroxypropionitrile

(76) Inventors: Seong-Jin Kim, Daejeon (KR); Jeong-Ho Song, Daejeon (KR); Jin-Won Yun, Daejeon (KR); Ho-Seong Lee, Daejeon (KR); Ho-Cheol Kim, Daejeon (KR)

Correspondence Address: HOFFMANN & BARON, LLP, 6900 JERICHO TURNPIKE, SYOSSET, NY, 11791, US

(21) Appl. No.: 10/592,223

(22) PCT Filed: Mar. 13, 2004
(86) PCT No.: PCT/KR04/00531

§ 371 Date: Sep. 8, 2006

(51) Int. Cl. C07C 253/16

(52) U.S. Cl. 558/309

(57) ABSTRACT

The present invention relates to a method for the preparation of 3-substituted-3′-hydroxypropionitrile, more particularly, to a method for the preparation of 3-substituted-3′-hydroxypropionitrile which comprises performing ring opening of 1-substituted-ethylene oxide using sodium cyanide and citric acid in a range of pH 7.8˜8.3 to provide 3-substituted-3′-hydroxypropionitrile in high optical purity and with high yield.

### TECHNICAL FIELD OF THE INVENTION

The present invention relates to a method for the preparation of 3-substituted-3′-hydroxypropionitrile, more particularly, to a method for the preparation of 3-substituted-3′-hydroxypropionitrile by ring opening of an epoxy compound using a cyanide group.

### BACKGROUND OF THE INVENTION

3-substituted-3′-hydroxypropionitrile is an essential key intermediate used in the preparation of medicals, agricultural products, bio-products and fine chemicals, such as L-carnitine, (S)-3-hydroxytetrahydrofuran or (S)-1,2,4-butantriol which is raw material of anti-cancer agents, ethyl (S)-chloro-3-hydroxybutyrate or ethyl (R)-cyano-3-hydroxybutyrate which is raw material of atorvastatin. Specifically, the ethyl (S)-chloro-3-hydroxybutyrate can be easily prepared from ethyl (S)-4-chloro-3-hydroxybutyronitrile which is a specific exemplary 3-substituted-3′-hydroxypropionitrile, by adding gaseous HCl supplied from HCl generator or HCl gas storage to a solution of the ethyl (S)-4-chloro-3-hydroxybutyrate in anhydrous ethanol.

Generally, the 3-substituted-3′-hydroxypropionitrile has been prepared through a ring opening reaction of an epoxy compound with a cyanide group:

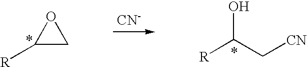

The ring opening reaction of the epoxy compound can be carried out in various conditions including an acid, a base and a solvent. Reaction yield markedly depends on the reaction condition. Particularly, when an optically active species is used as a staring material, optical purity much highly depends on the reaction condition.

Various methods for the production of the 3-substituted-3′-hydroxypropionitrile are known from the literatures. Bull. Soc. Chim. Fr. 3, 138(1936), Bull. Acad. R. Belg. 29, 256(1943), Ber., 12, 23(1879) or Japanese published patent No. H11-39559 discloses a method in which HCN is used. However, HCN is a toxic material such that handling of HCN is very dangerous process. Japanese published patent No. S63-316758 performs ring opening reaction using cyanide salt under a condition that acetic acid is used and reaction media is maintained in a range of pH 8.0˜10.0. However, yield and purity were found to be so low that the method can not be applicable to industrial production. As an improvement of the method, Japanese published patent No. H05-310671 discloses a method in which a cyanide salt and an inorganic acid was used and pH is maintained in a range of 8.0˜10.0. While this method is believed to be the best process providing high economic efficiency, it also suffers from the disadvantages that use of highly concentrated sulfuric acid and potassium cyanide threatens safety of workers, filtering process of inorganic salt is additionally required, and as shown in Example 16 of the specification of the Japanese unpublished patent No. H05-310671, the configuration of achiral starting material is not retained, which reduces optical purity of the product.

### SUMMARY OF THE INVENTION

Our inventers have strongly studied to establish improved conditions of the epoxy ring opening reaction which provides an increased yield and safe working environment and, particularly, retained optical purity of the raw material. As a result, we found an optimized condition of the epoxy ring opening reaction which provides sufficiently enhanced yield and retained optical purity of the raw material, in which citric acid and sodium cyanide which are not toxic and easily treatable were used, and pH was maintained in a range of 7.8˜8.3.

Therefore, the object of the present invention is to provide an improved method in which 3-substituted-3′-hydroxypropionitrile is produced in a high optical purity and chemical purity, and with high yield.

### DETAILED DESCRIPTION OF THE INVENTION

The present invention relates to a method for the preparation of 3-substituted-3′-hydroxypropionitrile by ring opening of an epoxy compound with a cyanide group, wherein the source of the cyanide group is sodium cyanide and the sodium cyanide is added together with citric acid to a reaction solution containing the epoxy compound, and pH of the reaction solution is maintained in a range of 7.8˜8.3. Preferably, the 3-substituted-3′-hydroxypropionitrile and the epoxy compound has formula 1 and 2, respectively:

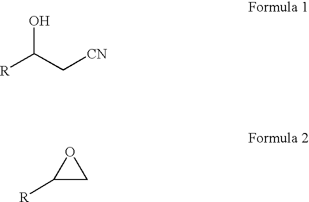

wherein, R represents C₁˜C₁₀ alkyl group, C₂˜C₆ alkenyl group, C₂˜C₆ alkynyl group, C₃˜C₈ cycloalkyl group, C₁˜C₁₀ alkoxy group, phenyl group, carbonyl group, carboxyl group, ketone group, aldehyde group, ester group, phosphoryl group, phosphonate group, phosphine group, sulfonyl group or —(CH₂)ₗ—R¹; R¹ represents C₂˜C₆ alkenyl group, C₂˜C₆ alkynyl group, C₂˜C₆ alkoxy group, phenyl, cycloalkyl, cycloalkenyl, heterocycle or polycycle, halogen atom, hydroxyl group, amino group, thiol group, nitro group, amine group, imine group, amide group, carbonyl group, carboxyl group, silyl group, ether group, thioether group, selenoether group, ketone group, aldehyde group, ester group, phosphoryl group, phosphonate group, phosphine group, sulphonyl group, and l is an integer of 0 to 8.

The ring opening reaction of an epoxy compound with a cyanide group can be summarized in a reaction scheme 1:

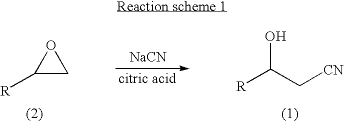

wherein, R is as defined in formula 1.

Detailed illustration of the present invention is as follows. The epoxy compound having formula 2 reacts with a cyanide group of sodium cyanide and produces 3-substituted-3′-hydroxypropionitrile having formula 1. Herein, the reaction is carried out in an aqueous system, the sodium cyanide is added together with citric acid, and pH of the reaction solution is maintained in a range of 7.8˜8.3.

According to the method of the present invention, it is important to maintain pH of the reaction media in a range of 7.8˜8.3, preferably 7.9˜8.2, because higher pH produces much more by-products and deteriorates reaction yield, and lower pH also produces somewhat more by-products and elongates reaction time. In order to adjust the pH to the above mentioned range, various acids might be adopted. According to the present invention, citric acid which is a tri-acid having three carboxyl group was used, and this is one of technically distinguishing points of the present invention. The citric acid easily dissolves in water solvent so that it can be used as a concentrated solution, which gives another industrial advantage. Further, the citric acid has no reactivity with the targeted product obtained from the ring opening reaction, thereby producing no byproduct which might be produced from the reaction of the citric acid with the targeted product. To the contrary, acetic acid, which is one of organic acids, produces as a byproduct 3-substituted-3′-acetoxypropionitrile which is obtained from the addition of the citric acid to the hydroxyl group of the targeted product, and the 3-substituted-3′-acetoxypropionitrile has a similar boiling point with the targetcd product, which makes it difficult to remove the byproduct through distillation purification. For these reasons, use of the acetic acid instead of the citric acid would give reduced purity of the targeted product and has a limit to apply to industrial production.

As illustrated in the above, the citric acid according to the present invention yields little byproduct and is useful for the preparation of the target product in a high chemical purity. Particularly, the citric acid has somewhat higher pKa value than inorganic acids such that it makes it possible to retain the optical purity of the starting material and to produce the chiral product in a high optical purity.

According to the characteristic of the present invention, sodium cyanide is added together with the citric acid. Among various cyanide salts, the sodium cyanide has the lowest toxicity and provides easy handing. The sodium cyanide is used in a range of 1.0˜2.0 equivalents, preferably 1.3˜1.5 equivalents.

The epoxy ring opening reaction of the present invention is carried out in a temperature of 10˜30° C., preferably 20˜25° C.

When the ring opening reaction is completed, an organic solvent is added to the reaction bottle to extract the targeted product, the organic solvent is removed by evaporation, and then, the residue was distilled to give the targeted 3-subtituted-3′-hydroxypropionitrile. This simple workup process is also one of the advantages of the present invention.

Japanese published patent No. S63-316758 and H05-301671 which are prior arts of the prevent invention suffers from the disadvantages of low yield, dangerous working environment, requirement of additional filtration of the inorganic salt, and particularly no retention of optical configuration of the starting material. To the contrary, the method according to the present invention is an improved process and provided enhanced effects in that: the method has an industrially advantageous reaction condition due to the use of the citric acid and the sodium cyanide which are no harmful and easily treatable; the method includes a simple workup process; and the method provides the targeted 3-subtituted-3′-hydroxypropionitrile represented in formula 1 in a high optical purity and chemical purity, and with a high yield.

In the following, the present invention will be more fully illustrated referring to Examples, but it should not be construed that the scope of the present invention is limited thereto.

### EXPERIMENTAL EXAMPLE 1

Preparation of (S)-4-chloro-3-hydroxybutyronitrile

3.75 L of water was added to (S)-epichlorohydrin (99.3% ee), and while stirring 4.76 kg of 25% aqueous sodium cyanide solution and 3.30 kg of 50% aqueous citric acid solution were dropped for 1 h 50 min to the solution under maintaining the reaction condition to pH 7.9˜8.2 and 22˜25° C. After additional 10 h stirring, 0.7 kg of sodium chloride was added and dissolved. The resulting solution was extracted with 20 L of ethyl acetate. 0.2 Kg of anhydrous sodium sulphate was added to the ethyl acetate layer, and then the solution was stirred for 30 min and filtered. The ethyl acetate was evaporated under reduced pressure, and the residue was distilled with thin film distillator (110° C./1 mbar) to give 1.77 kg of the targeted compound (yield 91.3%, chemical purity 99.1%).

Optical purity(GC)=99.3% ee

In the same manner as described in the Experimental Example 1, the reaction was performed under various conditions summarized in Table 1:

TABLE 1

| | | reaction | | chemical | optical |
|---|---|---|---|---|---|
| acid | pH | time | yield | purity | purity |
| citric | 7.9-8.2 | 10 h | 91.3% | 99.1% | 99.3% ee |
| acid | | | | | |
| citric | 8.5-8.7 | 7 h | 84.9% | 95.0% | 99.3% ee |
| acid | | | | | |
| citric | 7.5-7.7 | 23 h | 86.3% | 94.0% | 99.3% ee |
| acid | | | | | |
| acetic | 7.9-8.2 | 19 h | 75.8% | 89.0% | 99.2% ee |
| acid | | | | | |
| Sulphuric | 7.9-8.2 | 9 h | 82.0% | 99.0% | 98.7% ee |
| acid | | | | | |

### EXPERIMENTAL EXAMPLE 2

Preparation of (R)-4-chloro-3-hydroxybutyronitrile

The reaction was performed in the same manner as described in the experimental Example 1 except that (R)-epichlorohydrin (99.5% ee) was used instead of (S)-epichlorohydrin. As a result, the targeted compound was obtained: chemical purity 99.1%; optical purity (GC) 99.5% ee; and yield 91.3%.

### EXPERIMENTAL EXAMPLE 3

Preparation of Racemic 4-chloro-3-hydroxybutyronitrile

The reaction was performed in the same manner as described in the experimental Example 1 except that racemic epichlorohydrin was used instead of (S)-epichlorohydrin. As a result, the targeted compound was obtained: chemical purity 99.1% and yield 91.4%.

### EXPERIMENTAL EXAMPLE 4

Preparation of Chiral 3-substituted-3′-hydroxypropionitrile

0.36 L of water was added to 1.62 mole of each of the chiral epoxy compounds (>99% ee) listed in Table 2, and while stirring 476 g of 25% aqueous sodium cyanide solution and 330 g of 50% aqueous citric acid solution were dropped for 1 h to the solution under maintaining the reaction condition to pH 7.9˜8.2 and 22˜25° C. After stirring at room temperature, 70 g of sodium chloride was added and dissolved. The resulting solution was extracted with 2 L of ethyl acetate. 20 g of anhydrous sodium sulphate was added to the ethyl acetate layer, and then the solution was stirred for 30 min and filtered. The ethyl acetate was evaporated under reduced pressure. Fractional distillation under high vacuum of the residue gave the targeted compound in a chemical purity >99%, optical purity >99% ee, and yield >99%.

TABLE 2

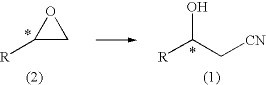

| R | optical purity(% ee) |
|---|---|
| Et | >99% ee |
| n-Bu | >99% ee |
| Cyclohexyl | >99% ee |
| Benzyl | >99% ee |
| t-Bu | >99% ee |
| CF₃CH₂ | >99% ee |
| Ph | >99% ee |
| 3-Butenyl | >99% ee |
| CH₃CO | >99% ee |
| PhOCH₂ | >99% ee |
| EtO₂CCH₂ | >99% ee |

As illustrated in the above, the method according to the present invention provides 3-substituted-3′-hydroxypropionitrile in high optical and chemical purities. Further, it employs citric acid and sodium cyanide which are no harmful and easily treatable such that an industrially advantageous reaction condition can be achieved, and it has simple work up process and high yield such that cost saving process in the preparation of can be achieved. Therefore, the method according to the present invention is useful for the preparation of 3-substituted-3′-hydroxypropionitrile in an industrial scale.

1. A method for preparing 3-substituted-3′-hydroxypropionitrile by ring opening of an epoxy compound with a cyanide group, wherein the source of the cyanide group is sodium cyanide and the sodium cyanide is added together with citric acid to a reaction solution containing the epoxy compound, and pH of the reaction solution is maintained in a range of 7.8˜8.3.

2. The method of claim 1, wherein the ring opening is performed in an aqueous system.

3. The method of claim 1, wherein the epoxy compound has a formula 2: 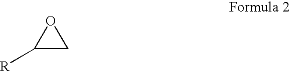
wherein, R represents C₁˜C₁₀ alkyl group, C₂˜C₆ alkenyl group, C₂˜C₆ alkynyl group, C₃˜C₈ cycloalkyl group, C₁˜C₁₀ alkoxy group, phenyl group, carbonyl group, carboxyl group, ketone group, aldehyde group, ester group, phosphoryl group, phosphonate group, phosphine group, sulfonyl group or —(CH₂)ₗ—R¹; R¹ represents C₂˜C₆ alkenyl group, C₂˜C₆ alkynyl gitoup, C₂˜C₆ alkoxy group, phenyl, cycloalkyl, cycloalkenyl, heterocycle or polycycle, halogen atom, hydroxyl group, amino group, thiol group, nitro group, amine group, imine group, amide group, carbonyl group, carboxyl group, silyl group, ether group, thioether group, selenoether group, ketone group, aldehyde group, ester group, phosphoryl group, phosphonate group, phosphine group, sulphonyl group, and l is an integer of 0 to 8.

4. The method of claim 1, wherein the epoxy compound is chiral.

5. The method of claim 1, comprising:
(a) performing ring opening of an epoxy compound to produce 3-substituted-3′-hydroxypropionitrile, wherein the ring opening is performed in an aqueous system, the source of the cyanide group is sodium cyanide, the sodium cyanide is added together with citric acid to a reaction solution containing the epoxy compound, and pH of the reaction solution is maintained in a range of 7.8˜8.3;
(b) extracting the produced 3-substituted-3′-hydroxypropionitrile with an organic solvent,
(c) evaporating the extracted solution to remove the organic solvent, followed by distillation to obtain the targeted 3-substituted-3′-hydroxypropionitrile.
